(19) United States
(12) Patent Application Publication
(10) Pub. No.: US 2020/0246061 A1
(43) Pub. Date: Aug. 6, 2020

(54) RESECTOSCOPE WITH ILLUMINATION MODE FOR SUPPORTING ELECTROSURGICAL COAGULATION

(71) Applicant: OLYMPUS WINTER & IBE GMBH, Hamburg (DE)

(72) Inventor: Christoph KNOPF, Lübeck (DE)

(73) Assignee: OLYMPUS WINTER & IBE GMBH, Hamburg (DE)

(21) Appl. No.: 16/775,496

(22) Filed: Jan. 29, 2020

(30) Foreign Application Priority Data
Feb. 5, 2019 (DE) 102019102838.8

(51) Int. Cl. A61B 18/14; A61B 1/06; A61L 31/02; A61B 1/07; A61B 1/00

(52) U.S. Cl. CPC A61B 18/149; A61B 1/063; A61B 2018/00982; A61B 1/07; A61B 1/00066; A61L 31/022

(57) ABSTRACT

A resectoscope for endoscopic surgery with a tubular shaft and a handle, wherein the shaft includes a longitudinally displaceable electrode instrument and an illuminating device, with an electrode to which high-frequency current can be applied being arranged in the distal end region of the electrode instrument, characterized in that the illuminating device in the distal end region of the resectoscope can emit UV radiation in the wavelength range from 200 nm to 400 nm.

### BACKGROUND

The invention relates to resectoscopes for endoscopic surgery of the type mentioned in the preamble of claim 1.

Resectoscopy systems of this generic type are used above all in urology for surgical work in the bladder and in the urethra. They are usually used for resection and vaporization of tissue, for example tissue in the lower urinary tract. For this purpose, the resectoscopes used comprise a longitudinally displaceable electrosurgical electrode instrument which, after insertion of the resectoscope, can be pushed with its distal working end out of the distal end of the shaft tube of the resectoscope. In addition, resectoscopes for observing the site of intervention and monitoring the intervention generally contain an optical system which comprises an objective at the distal end and is connected to an eyepiece for direct observation or to an electronic monitoring unit at the proximal end. Finally, the resectoscopes for illuminating the site of intervention contain an illuminating element, usually in the form of an optical fiber bundle, that spans the shaft of the resectoscope and is connected to a light source at its proximal end.

The electrode instrument that is passed through the resectoscope shaft can comprise an electrosurgical electrode in the form of a Loop or PlasmaButton at its distal working end. Examples of such instruments include the OES PRO resectoscopes (Olympus). The electrode instruments can be embodied as bipolar or monopolar instruments, although bipolar instruments can have substantial safety advantages over monopolar instruments.

At present, bipolar electrodes are usually made of 1.4301 or 1.4542 stainless steel.

During an operation, a glow discharge or a plasma is generated at the electrode in which hydroxyl radicals (OH.) are generated, which contribute significantly to tissue separation or tissue coagulation. Due to their reactivity, these primarily formed hydroxyl radicals have only a small effective radius. However, they react in the gas phase to form hydrogen peroxide (H₂O₂), which is less reactive and therefore has a larger effective radius. Using the metal ions that are dissolved out of the electrode—such as iron ions (Fe³⁺), for example—the hydrogen peroxide can then be broken down again by a “Fenton-like reaction”: Fe²⁺+H₂O₂→Fe³⁺+OH.+OH⁻. The hydroxyl radicals that are generated again in this way can again contribute to tissue separation or tissue coagulation.

However, the effectiveness of the hydroxyl radical generation described above is adversely affected by the fact that the hydroxyl ions (OH⁻) bind free metal ions, e.g., iron ions (Fe³⁺): Fe³⁺+OH⁻↔Fe(OH)²⁺. The metal ions bound in this way can therefore not contribute to the splitting of further hydrogen peroxide, so the coagulating or separating action of the electrode on the tissue is limited.

It is therefore the object of the present invention to provide an even further optimized resectoscope in which the coagulating and separating effect of the electrode on surrounding tissue is enhanced.

### DESCRIPTION

This object is achieved by a resectoscope with the features of claim 1.

In a first aspect, the invention relates particularly to a resectoscope for endoscopic surgery with a tubular shaft and a handle, wherein the shaft comprising a longitudinally displaceable electrode instrument and an illuminating device, with an electrode to which high-frequency current can be applied being arranged in the distal end region of the electrode instrument, characterized in that the illuminating device in the distal end region of the resectoscope can emit UV radiation in the wavelength range from 200 nm to 400 nm.

Through irradiation of the electrode and the site of intervention with light in the UV range, the metal ions bound by means of the reaction described above into Fe(OH)²⁺, for example, are photoreduced after the following exemplary reaction and released to form hydroxyl radicals (OH.): Fe(OH)²⁺+hv→Fe²⁺+OH.. The reaction proceeds analogously with other metal ions. The principle according to the invention can thus also be applied to metallic electrodes that do not contain iron. The coagulating or separating effect of the electrode on the tissue can thus be substantially increased by means of the inventive irradiation with UV light.

In a common design, the resectoscope has a tubular shaft which, according to the invention, comprises an illuminating device. The illuminating device is suitable for emitting UV radiation—i.e., radiation with wavelengths in the UV range—at its distal end, preferably in the distal direction. In a preferred embodiment, the illuminating device can emit UV radiation in the wavelength range from 200 nm to 400 nm in the distal end region of the resectoscope. In other words, the illuminating device configured to emit UV radiation in the aforementioned wavelength range. In some embodiments, the illuminating device can emit UV radiation over this entire wavelength range, whereas it can emit UV radiation only in one or more subranges of this wavelength range in other embodiments.

The UV range comprises wavelengths from 1 nm to 400 nm, it being preferred according to the invention that the UV radiation used have wavelengths from 200 to 400 nm, since shorter wavelengths could have an undesirable ionizing effect on the tissue. Since it is to be expected that wavelengths in the visible violet range (from 400 nm) have diminished effectiveness, it is also preferred according to the invention that the illuminating device emit wavelengths in the range from 200 to 380 nm. In other words, it is preferred that the UV radiation be selected from the group consisting of the near-UV (315 nm to 380 nm), middle-UV (280 nm to 315 nm) and far-UV ranges (200 nm to 280 nm); radiation in the middle- and far-UV range is particularly preferred, and radiation in the middle UV range is even more preferred. It is also envisaged that the illuminating device not emit any or that it emit less than 10%, preferably less than 5% of its power in the UV range from 1 nm to 200 nm.

In some embodiments, the illuminating device can also emit radiation in the visible range in addition to the radiation in the UV range. In this embodiment, the illuminating device can be used for simultaneously illuminating the area of intervention with visible light for visual monitoring by means of optics and irradiating the area of intervention with UV radiation to improve the coagulating or separating effect of the electrode on the tissue. However, it is also conceivable to separate these functions and to provide a second illuminating device for irradiating the engagement area with visible light in the shaft. The radiation emitted by the second illuminating device would then have one or more radiation power peaks in the wavelength range of visible light—i.e., from 400 nm to 780 nm.

According to the invention, in addition to the radiation in the wavelength ranges that are visible to humans, the term “light” also includes radiation in the UV wavelength range, particularly in the wavelength range from 200 nm to 400 nm. In the present document, “visible” light means wavelengths that are visible to humans—i.e., wavelengths in the range from approximately 400 nm to 780 nm.

It will readily be understood that the proportion of UV radiation in the radiation emitted by the illuminating device is significant, meaning that, in relation to the other radiation emitted by the illuminating device, it is not limited only to a low residual radiation power. The illuminating device according to the invention will generally have one or more radiant flux peaks in the UV wavelength range, preferably from 200 nm to 400 nm, more preferably from 200 nm to 380 nm, based on the wavelength spectrum of the radiation emitted by the illuminating device. In especially preferred embodiments, the illuminating device has one or more radiant flux peaks in the wavelength range from 280 nm to 315 nm (middle UV radiation). As described above, the illuminating device can also be used to illuminate the area of the intervention. There can thus be one or more additional radiant flux peaks, for example in the wavelength range of visible light.

The illuminating device can comprise or consist of an optical fiber bundle that is arranged in the shaft. The optical fiber bundle can pass through the elongate shaft of the resectoscope. At the distal end, the optical fiber bundle emits in the desired direction, and the optical fiber bundle is connected to a light source at the proximal end. The optical fiber bundle can have a circular cross section or a cross section of another shape, for example a substantially half-moon or irregular shape.

Alternatively, the illuminating device can comprise a diode in the distal end region of the resectoscope. The diode is preferably a light emitting diode (LED). When electrical current flows through the diode, it emits light. The diode is supplied with current via a power line running in the shaft. Compared to the optical fiber bundles described elsewhere, the power line has a substantially smaller cross-sectional diameter. This significantly increases the space available for holding devices in the shaft.

The illuminating device is configured to emit UV radiation at its distal end, i.e., at the distal end of the resectoscope. The illuminating device can emit in the distal direction and/or in the direction of the electrode and/or in the direction of the site of intervention. In a first embodiment, the illuminating device emits in the distal direction. In an alternative embodiment, the illuminating device emits at an angle to the longitudinal axis of the shaft in the direction of the electrode. As a result, the area to be illuminated can be kept small, and the risk of damage to the tissue caused by UV radiation can be reduced to a minimum. However, it is also conceivable for the illuminating device according to the invention to irradiate a larger area, particularly if the illuminating device is also used to illuminate the site of the intervention with visible light.

The direction of radiation can be variable (i.e., adjustable) or fixed (i.e., not adjustable). For example, it is conceivable for the direction of radiation to be made adjustable by means of an actuating device that can be arranged on the handle, for example. In this way, the direction of radiation could be flexibly set to the position of the electrode when the electrode instrument is moved in the longitudinal direction of the shaft. The light emitted by the illuminating device, particularly the UV radiation emitted by the illuminating device, can thus be preferably directed in the direction of the electrode.

The resectoscope according to the invention also has a handle. An actuating device for actuating and/or controlling the illuminating device can be arranged on this handle, for example. The actuating device can be designed to switch the illuminating device on and off. Alternatively or in addition, the radiation intensity can be varied by means of the actuating device. In addition, it is conceivable that the direction of radiation of the illuminating device can be varied by means of the same or a separate actuating device.

As already mentioned elsewhere, the shaft of the resectoscope comprises an electrode instrument that is arranged so as to be displaceable longitudinally. The electrode instrument has an elongate shaft part and is embodied as a pass-through instrument for a resectoscope, i.e., as an instrument that can be inserted into a body opening through a resectoscopic shaft tube. At its distal end, the electrode instrument has an electrode to which high-frequency current can be applied. The electrode can be a cutting Loop, a PlasmaButton, or other commercially available electrodes. The electrode is preferably a cutting loop electrode. Such electrodes and electrode instruments are known to those skilled in the art.

The electrode instrument can be a bipolar electrode instrument that includes the electrode as part of an electrode assembly. In that case, the electrode instrument will, for example, comprise a second electrode in the distal end region of the electrode instrument that is embodied as a neutral electrode. Alternatively, the second electrode (neutral electrode) can also be arranged on other elements of the distal end region of the resectoscope. As will readily be understood, the resectoscope can also be designed as a monopolar instrument.

According to the invention, the electrode comprises or consists of an electrically conductive, preferably metallic material. The material can be selected from the group consisting of: Metals of the 4th period of the periodic table (Sc, Ti, V, Cr, Mn, Fe, Co, Ni, Cu, Zn) and metals of subgroup VI of the periodic table (Cr, Mo, W, Sg), as well as alloys that comprise the above materials. Preferably, the material is a steel, more preferably a stainless steel. Stainless steels are steels whose sulfur and phosphorus content is less than 0.025%. The steel used is suitable for use in surgical instruments. Steels suitable for this use are known to those skilled in the art and described in ISO 7153-1, for example. In the context of the invention, the electrode thus comprises or consists in particular of a steel described in ISO 7153-1. The electrode especially preferably comprises iron. For example, the electrode can comprise a steel that is selected from the group consisting of: 1.4301 (according to DIN: X5CrNi18-10) and 1.4542 (according to DIN: X5CrNiCuNb17 44), or it can be composed of one of these steels.

The electrode instrument is longitudinally displaceable within the shaft of the resectoscope, meaning that it can be moved distally and proximally in the axial direction. For connection to the resectoscope, the electrode instrument has an elongate shaft, which can be fastened at its proximal end to a slide that is encompassed by the resectoscope in order to produce a movement-coupled connection. The slide typically slides on a tube and is spring biased by a spring unit into a rest position. The electrode at the distal end can thus be moved toward or away from tissue to be resected without the need to move the entire resectoscope. Moreover, the longitudinal displaceability of the electrode instrument makes it possible to clamp tissue between the electrode and the distal end of the inner tube and remove it from the site of intervention. The distal end of the inner tube and the electrode can thus be moved toward and away from one another by virtue of the longitudinal displaceability of the electrode instrument.

The resectoscopes according to the invention can be used in all areas of endoscopic surgery. They are particularly well suited for use in narrow body canals such as the urethra. For this purpose, the resectoscopes have the already-described tubular shaft. In the usual manner, the shaft can have an outer tube and an elongate inner tube that extends through the outer tube, with the illuminating device that is used according to the invention preferably being arranged in the inner tube. For example, the illuminating device can be arranged in the inner tube next to the shaft section of the electrode instrument, for example between or under the fork tubes of the electrode instrument. In this arrangement, the illuminating device can easily illuminate the electrode and the area of the intervention with UV radiation.

The resectoscope according to the invention also has optics, i.e., an optical image bundle, for viewing the area of intervention and monitoring the intervention. The optics run through the shaft over its length and, as mentioned above, are also arranged in an inner tube, for example. The optics can comprise an ordered fiber bundle and/or rod lenses arranged one behind the other. The optics have an objective lens at their distal end and an eyepiece at their proximal end. The viewer's eye looks through the optics at an observation area that lies in front of the distal end face of the shaft. Alternatively, the optics can also be connected to a digital imaging unit at their proximal end. Particularly in embodiments in which the illuminating device is used in addition to illuminating the site of intervention with visible light, it is advantageous if the optics are arranged in the shaft directly next to the shaft part of the illuminating device. In this arrangement, the illuminating device can illuminate the area of the intervention that can be monitored by means of the optics with visible light.

The direction of radiation of the illuminating device and the viewing direction of the optics can be coordinated with one another. In this way, it is ensured that the electrode is irradiated with UV radiation over its entire stroke. The direction of radiation of the illuminating device is thus preferably essentially identical to the viewing direction of the optics of the resectoscope. The “direction of radiation” of the illuminating device is understood here to be the direction in which the illuminating device emits with maximum luminous intensity (100%). As a rule, this direction corresponds to the direction of the longitudinal axis of the illuminating device in its distal end region. In other words, the viewing angle (α) of the optics relative to the longitudinal axis of the resectoscope is preferably identical to the viewing angle (β) of the illuminating device relative to the longitudinal axis of the resectoscope. Moreover, it is also envisaged that the area that is irradiated by the illuminating device be coordinated with the size of the area that is visible with the optics. The two areas can have a common center point, for example. In addition, particularly also the radiation angle of the illuminating device—i.e., the angle that is formed by the lateral points with half the maximum luminous intensity—can be selected such that the illuminating device can completely illuminate the field of view of the optics with half the maximum luminous intensity or more. It is also conceivable for the illuminating device and the optics—particularly the direction of radiation or viewing direction thereof—to be synchronized automatically with one another.

It is conceivable for individual components that pass through the shaft part of the resectoscope to be stabilized against one another, particularly counter to a displacement in the radial direction.

The electrode instrument preferably has guide elements that serve to support and stabilize the electrode instrument within the inner tube. For this purpose, the guide elements adjoin the inner wall of the inner tube in such a way that movement of the electrode instrument in the axial direction and potentially also rotational movements about the longitudinal axis are possible, while movements of the electrode instrument in the radial direction are reduced or prevented. It has been found to be especially advantageous for the guide elements to be partially complementary in shape to the inner wall. The guide elements can have a partially circular cross section, for instance. Such guide elements are known to those skilled in the art. The guide elements can be made of metal or other materials. The guide elements are especially preferably guide plates. As a rule, no further parts are arranged between the electrode instrument, or the guide elements thereof, and the inner wall of the inner tube. Further components, such as an optical system, for example, can be arranged within the inner tube, however.

Similarly, one or more stabilizing elements can be arranged between the optics and the illuminating device which stabilize these two elements against radial displacement. The optics and the illuminating device can also be secured in this way against displacement in the longitudinal direction. For this purpose, the stabilizing elements can have shapes analogous to those of the guide elements described above. The stabilizing elements can thus have two partially circular portions, the first being complementary in shape and size to the outer wall of the optics and the second being complementary in shape and size to the exterior of the illuminating device.

In a second aspect, the invention relates to an electrosurgical method that comprises steps in which a) a resectoscope according to the invention is provided; b) a tissue in a body opening or surgical opening of a patient is irradiated with UV radiation, the UV radiation preferably being emitted by an illuminating device of the resectoscope; and c) during the illumination of the tissue with UV radiation with an electrode of the resectoscope, the tissue is resected and/or coagulated.

### BRIEF DESCRIPTION OF THE FIGURES

Exemplary embodiments of the invention are shown schematically in the drawings, in which:

FIG. 1 shows a sectional view of a resectoscope according to the invention with an electrode instrument that can be moved longitudinally in the shaft and has an electrode at its distal end; and

FIG. 2 shows a front view of the shaft of the resectoscope according to the invention shown in FIG. 1.

### EXEMPLARY EMBODIMENTS

Additional advantages, characteristics, and features of the present invention will become clear from the following detailed description of exemplary embodiments with reference to the attached drawings. However, the invention is not restricted to these exemplary embodiments.

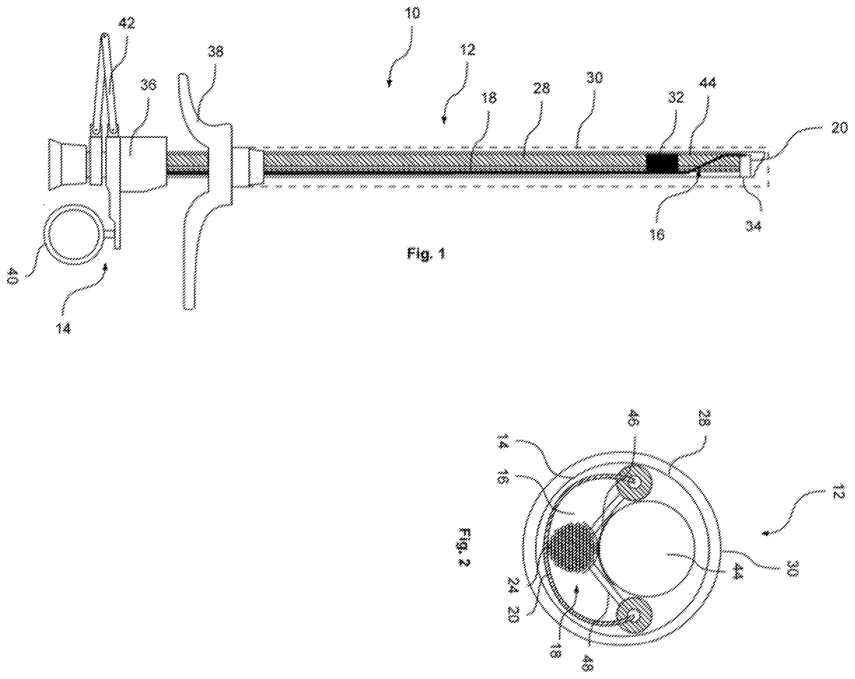

FIGS. 1 and 2 show a sectional view of a resectoscope 10 according to the invention and a front view of the shaft 12 of this resectoscope 10. The resectoscope 10 has an electrode 20, which is embodied as a loop electrode, and an illuminating device 18, which comprises an optical fiber bundle 24.

The illuminating device 18 can emit radiation in the UV range in the distal end region of the resectoscope 10. In particular, a provision is made that the illuminating device 18 emits UV radiation between 200 nm and 400 nm. In addition to a radiant flux peak in this range between 200 nm and 400 nm, the illustrated illuminating device 18 has an additional radiant flux peak in the range of light that is visible to humans. Therefore, in the illustrated embodiment, it is not necessary to provide a second illuminating device in order to illuminate the site of intervention.

As can be seen in FIG. 1, the resectoscope 10 has a shaft 12 that comprises a cladding tube 30 (outer tube), which is shown in broken lines. An inner tube 28 runs inside the cladding tube 30, and the electrode instrument 16, the optics 44, and the illuminating device 18 run inside the inner tube 28. Furthermore, additional elements such as an irrigation tube or a second illuminating device (not shown here) can run inside the cladding tube 30, insofar as the lighting with visible light is to be provided separately from the irradiation with UV light.

In the embodiment shown here, the optics 44 and the illuminating device 18 are supported against one another and secured against radial displacement and displacement in the longitudinal direction of the shaft 12 by means of a stabilizing element 46. The stabilizing element 46 has two portions that are fastened to one another, each with a partially circular cross section, one of which is complementary in shape and size to the outer wall of the optics and one which is complementary in shape and size to the outside of the optical fiber bundle 24.

The electrode instrument 16 is arranged so as to be longitudinally displaceable in the inner tube 28 and is protected against transverse displacements—i.e., displacements in the radial direction—by a holding element 32 (guide element). The holding element 32 is complementary in shape to the inner wall of the inner tube 28 or to the outer wall of the optics 44 and has a partially cylindrical shape. The holding element 32 is fastened to two fork tubes 48 in a shaft portion of the electrode instrument 16. The fork tubes 48 run closely together within the shaft 12 and diverge only in the distal end region of the shaft 12 in order to receive and carry the loop electrode between their ends.

The electrode instrument 16 can be moved in an axially guided manner in the distal and proximal direction through actuation of a handle part 40. It can be pushed over the distal end of the inner tube 28 and the cladding tube 30. This enables the surgeon to manipulate tissue that is farther away from the resectoscope tip. For this purpose, the inner tube 28 and/or the electrode instrument 16 are also supported so as to be rotatable about their longitudinal axes. The electrode instrument 16 has at its distal end an electrode 20 that is embodied as a cutting loop and by means of which tissue can be removed by electrosurgical ablation. Here, a high-frequency electrical voltage is applied to the electrode 20 in order to cut tissue.

The resectoscope 10 shown has a passive transporter in which the slide 36 is displaced in the distal direction against the distal, first handle part 38 through a relative movement of the handle parts 38 and 40 that are arranged proximally from the shaft 12 against a spring force that is applied by a spring bridge 42. When the slide 36 is displaced in the distal direction against the handle part 38, the electrode instrument 16 is positively guided to the distal in a manner not shown. When the handle parts 38, 40 are released, the spring force generated by the spring bridge 42 forces the slide 36 back into its resting position, the shaft 12 and hence the electrode instrument 16 as well being pulled in the proximal direction. When the slide 36 is moved back, an electrosurgical intervention with the electrode instrument 16 can be carried out without manual force on the part of the surgeon—that is, passively.

The electrode instrument 20 that is arranged at the distal end of the electrode instrument 16 is a metallic electrode and is made of steel (stainless steel) of the alloy 1.4301. This is stainless steel with the short name X5CrNi18-10, available from specialty retailers. It is also conceivable for the electrode 20 to be made from steel of the alloy 1.4542. This is stainless steel with the short name X5CrNiCuNb17 44. As explained elsewhere, it is of course also possible to make the electrode 20 from other metallic compounds, particularly from other steels, in particular from stainless steels.

As can be seen in FIGS. 1 and 2, the resectoscope 10 according to the invention has an illuminating device 18. The illuminating device 18 has an elongate shaft part that runs through the shaft 12 of the resectoscope. The illuminating device 18 comprises an optical fiber bundle 24 with a substantially round cross section. Alternatively, it is also conceivable for the optical fiber bundle 24 to have a different cross section. The optical fiber bundle 24 comprises a plurality of individual optical fibers that transport light from the proximal end to the distal end of the shaft.

In the embodiment that is shown, the optical fiber bundle 24 is not surrounded by its own sleeve. The desired cross-sectional shape is secured by gluing the fibers. Alternatively, however, it is also conceivable for the optical fiber bundle 24 to be enclosed with a sheath, for example with a hose-shaped or tubular sheath.

The UV radiation emitted at the distal end by the illuminating device 18 releases iron ions that are bound as Fe(OH)²⁺ under simultaneous release of hydroxyl radicals according to the following reaction: Fe(OH)²⁺+hv→Fe²⁺+OH.. The iron ions released in this way are then again available for trapping further hydroxyl ions (OH⁻), while the hydroxyl radicals (OH.) can contribute to tissue resection or coagulation. This significantly increases the coagulating or severing effect of the electrode on the tissue to be resected or coagulated. As set out above, a diode such as an LED, for example, can also be provided at the distal end of the shaft 12 instead of an optical fiber bundle 24. This diode would only need to be connected to a proximal power source via a line, thereby enlarging the available space in the shaft 12.

Depending on the embodiment, the illuminating device 18 is connected at its proximal end to a light source (in the case of an optical fiber bundle 24) or to a power source (in the case of a diode-containing illuminating device 18) via a connector cable (not shown here).

Although the present invention has been described in detail with reference to the exemplary embodiments, it is obvious to those skilled in the art that the invention is not restricted to these exemplary embodiments, but rather that modifications can be made in such a way that individual features are omitted or other combinations of the individual features presented are realized, provided that the scope of protection of the appended claims is not exceeded. The present disclosure includes any and all combinations of the individual features presented.

### LIST OF REFERENCE SYMBOLS

10 resectoscope
12 shaft
14 handle
16 electrode instrument
18 illuminating device
20 electrode
24 optical fiber bundle
28 inner tube
30 cladding tube
32 holding element
34 insulating tip
36 slide
38 handle part
40 handle part
42 spring bridge
44 optics
46 stabilizing element
48 fork tube

1. A resectoscope for endoscopic surgery with a tubular shaft and a handle, wherein the shaft comprising a longitudinally displaceable electrode instrument and an illuminating device, with an electrode to which high-frequency current can be applied being arranged in the distal end region of the electrode instrument, wherein the illuminating device in the distal end region of the resectoscope can emit UV radiation in the wavelength range from 200 nm to 400 nm.

2. The resectoscope as set forth in claim 1, wherein the electrode comprises a material that is selected from the group consisting of: metals of the 4th period of the periodic table (Sc, Ti, V, Cr, Mn, Fe, Co, Ni, Cu, Zn) and metals of subgroup VI of the periodic table (Cr, Mo, W, Sg), as well as alloys that comprise the above materials.

3. The resectoscope as set forth in claim 1, wherein the electrode comprises a steel that is suitable for use in surgical instruments.

4. The resectoscope as set forth in claim 1, wherein the wavelength spectrum of the radiation emitted by the illuminating device has one or more radiant flux peaks in the range from 200 nm to 400 nm.

5. The resectoscope as set forth in claim 1, wherein the wavelength spectrum of the radiation that is emitted by the illuminating device has one or more radiant flux peaks in the range from 200 nm to 315 nm.

6. The resectoscope as set forth in claim 1, wherein the illuminating device emits in the distal direction.

7. The resectoscope as set forth in claim 1, wherein the direction of radiation of the illuminating device is adjustable.

8. The resectoscope as set forth in claim 1, wherein the direction of radiation of the illuminating device is substantially identical to the viewing direction of an optical system of the resectoscope.

9. The resectoscope as set forth in claim 1, wherein the light emitted by the illuminating device can be guided in the direction of the electrode.

10. The resectoscope as set forth in claim 1, wherein the illuminating device comprises a diode in the distal end region of the resectoscope.

11. The resectoscope as set forth in claim 1, wherein the illuminating device comprises an optical fiber bundle that is arranged in the shaft.

12. The resectoscope as set forth in claim 1, wherein the handle comprises an actuating device for actuating and/or controlling the illuminating device.
